(19) United States
(12) Patent Application Publication
(10) Pub. No.: US 2020/0087582 A1
(43) Pub. Date: Mar. 19, 2020

(54) METHOD OF TREATING A HYDROCARBON STREAM

(71) Applicant: SABIC GLOBAL TECHNOLOGIES B.V., BERGEN OP ZOOM (NL)

(72) Inventors: Dafer Mubarak Alshahrani, Riyadh (SA); Shahid Azam, Riyadh (SA); Abdullah Saad Al-Dughaither, RIYADH (SA); Abdulmajeed Mohammed Al-Hamdan, Riyadh (SA)

(21) Appl. No.: 16/470,694

(22) PCT Filed: Dec. 19, 2017
(86) PCT No.: PCT/IB2017/058154

§ 371 Date: Jun. 18, 2019

Related U.S. Application Data
(60) Provisional application No. 62/436,173, filed on Dec. 19, 2016.

(51) Int. Cl. C10G 31/06; C10G 75/00; C07C 2/36; B01D 5/00; B01D 53/00; F25J 3/02

(52) U.S. Cl. CPC C10G 31/06; C10G 75/00; C07C 2/36; B01D 5/0027; B01D 5/0075; C10G 2300/1092; F25J 3/0219; C07C 2531/24; C10G 2300/4075; C10G 2400/22; C10G 2300/1088; B01D 53/002

(57) ABSTRACT

A method of treating a hydrocarbon stream includes: withdrawing an effluent stream comprising hydrocarbons and polymer from a reactor; contacting the effluent stream with a coolant stream; passing the effluent stream through a heat exchanger; wherein after passing the effluent stream through the heat exchanger, the heat exchanger is substantially free of polymer deposits.

### BACKGROUND

Linear alpha olefins are olefins or alkenes with a chemical formula CₓH₂ₓ, distinguished from other mono-olefins with a similar molecular formula by linearity of the hydrocarbon chain and the position of the double bond at the primary or alpha position. There are a wide range of industrially significant applications for linear alpha olefins. For example, the lower carbon numbers, 1-butene, 1-hexene and 1-octene can be used as co-monomer in the production of polyethylene.

Linear alpha olefins can often be produced via the oligomerization of ethylene. This process presents many engineering challenges. For example, an effluent stream withdrawn from an oligomerization reactor can comprise unreacted catalyst and by-products such as dissolved polymer. As a result, transfer of the effluent stream can result in the fouling of downstream piping and equipment. For example, a heat exchanger can often be employed to cool an effluent stream. The exchanger, downstream of the reactor, can become fouled with deposits of the dissolved polymer, thus reducing the efficiency of the exchanger and increasing maintenance requirements.

Thus, there is a need for a treatment method that can cool a hydrocarbon stream, reduce by-product formation within the hydrocarbon stream, and prevent polymer fouling of downstream piping and equipment.

### SUMMARY

Disclosed, in various embodiments, are methods of treating a hydrocarbon stream.

A method of treating a hydrocarbon stream, comprises: withdrawing an effluent stream comprising hydrocarbons and polymer from a reactor; contacting the effluent stream with a coolant stream; passing the effluent stream through a heat exchanger; wherein after passing the effluent stream through the heat exchanger, the heat exchanger is substantially free of polymer deposits.

A method of treating a hydrocarbon stream, comprising: withdrawing an effluent stream comprising hydrocarbons and polymer from a reactor, wherein the effluent stream comprises ethane, methane, ethylene, butane, hexene, toluene, octene, decene, catalyst particles, or a combination comprising at least one of the foregoing; directly mixing the effluent stream with a coolant stream, wherein the coolant stream comprises methane, ethylene, ethane, butane, hexene, or a combination comprising at least one of the foregoing and wherein the effluent stream is reduced in temperature by greater than or equal to 20% after contacting the coolant stream; passing the effluent stream through a pipe, wherein after passing the effluent stream through the pipe, the pipe is substantially free of polymer deposits; passing the effluent stream through a heat exchanger, wherein after passing the effluent stream through the heat exchanger, the heat exchanger is substantially free of polymer deposits; and withdrawing a product stream from the heat exchanger; wherein the source of the effluent stream and the coolant stream is a product of the same ethylene oligomerization process.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic diagram representing a method of treating a hydrocarbon stream.

### DETAILED DESCRIPTION

The method disclosed herein can reduce the temperature of a hydrocarbon effluent stream by greater than or equal to 20% and deactivate unreacted catalyst particles present within the stream. The method can significantly reduce the fouling of piping and downstream equipment caused by polymer and by-products present within the stream. The method can also improve the efficiency of downstream equipment, for example, reduce the duty of downstream heat exchangers by greater than or equal to 20%. The method can reduce overall capital costs, and the method can reduce the frequency and/or need for equipment maintenance.

The method disclosed herein can include withdrawing a gaseous hydrocarbon effluent stream from an oligomerization reactor and contacting the effluent stream with a coolant stream. The effluent stream can be reduced in temperature, thus creating a two-phase flow wherein liquid condensate droplets are formed in suspension within the gaseous effluent stream. The condensate droplets can then collide with, and dislodge, any polymer deposits present in the piping and downstream equipment. For example, downstream heat exchangers, employed to cool the effluent stream, can remain substantially free of polymer deposits and experience a reduction in both duty and maintenance requirements.

A feed stream to the present method can comprise a mixture of hydrocarbons. For example, the feed stream can comprise alkenes, for example, linear alpha olefins, for example, ethylene.

The feed stream can also comprise a catalyst, for example, a heterogeneous catalyst, for example, an oligomerization catalyst. For example, the catalyst can comprise a chromium compound and a ligand of the general structure (A) R₁R₂P—N(R₃)—P(R₄)—N(R₅)—H or (B) R₁R₂P—N(R₃)—P(R₄)—N(R₅)—PR₆R₇, wherein R₁-R₇ are independently selected from halogen, amino, trimethylsilyl, C₁-C₁₀-alkyl, C₆-C₂₀ aryl or any cyclic derivatives of (A) and (B), wherein at least one of the P or N atoms of the PNPN-unit or PNPNP-unit is a member of a ring system, the ring system being formed from one or more constituent compounds of structures (A) or (B) by substitution.

The feed stream can also comprise a solvent. For example, the solvent can comprise aromatic hydrocarbons, straight chain aliphatic hydrocarbons, cyclic aliphatic hydrocarbons, ethers, toluene, benzene, ethylbenzene, cumene, xylenes, mesitylene, hexane, octane, cyclohexane, methylcyclohexane, diethylether, tetrahydrofurane, or a combination comprising at least one of the foregoing.

The feed stream to the present method can be passed through a reactor. For example, the reactor can be a multi-phase reactor, a bubble column reactor, a slurry bed reactor, or a combination comprising at least one of the foregoing. For example, the reactor can be an oligomerization reactor. Accordingly, an oligomerization reaction can occur within the reactor, for example, an ethylene oligomerization reaction.

The oligomerization reaction can produce an effluent stream which can then be withdrawn from the reactor. For example, the effluent stream can be withdrawn from a top portion of the reactor. The effluent stream can be gaseous and/or liquid. The effluent stream can comprise hydrocarbons, such as linear alpha olefins, solvent and unreacted catalyst particles. For example, the effluent stream can comprise methane, ethylene, ethane, 1-butene, 1-hexene, toluene, 1-octane, 1-decene, 1-dodecene, catalyst particles, or a combination comprising at least one of the foregoing. For example, the effluent stream can comprise 0% to 5% 1-hexene. The effluent stream can comprise 0 to 50 parts per million (ppm) unreacted catalyst particles. The effluent stream can also comprise by-products, such as dissolved polymer, for example, greater than or equal to 1 ppm dissolved polymer. The dissolved polymer can be a by-product of the oligomerization reaction that occurs within the reactor. The effluent stream can have a temperature of greater than or equal to 45° C. when withdrawn from the reactor. The effluent stream can be transferred, via piping, from the reactor to subsequent downstream equipment.

1-Hexene is commonly manufactured by two general routes: (i) full-range processes via the oligomerization of ethylene and (ii) on-purpose technology. A minor route to 1-hexene, used commercially on smaller scales, is the dehydration of hexanol. Prior to the 1970s, 1-hexene was also manufactured by the thermal cracking of waxes. Linear internal hexenes were manufactured by chlorination/dehydrochlorination of linear paraffins.

“Ethylene oligomerization” combines ethylene molecules to produce linear alpha-olefins of various chain lengths with an even number of carbon atoms. This approach results in a distribution of alpha-olefins. Oligomerization of ethylene can produce 1-hexene.

Fischer-Tropsch synthesis to make fuels from synthesis gas derived from coal can recover 1-hexene from the aforementioned fuel streams, where the initial 1-hexene concentration cut can be 60% in a narrow distillation, with the remainder being vinylidenes, linear and branched internal olefins, linear and branched paraffins, alcohols, aldehydes, carboxylic acids, and aromatic compounds. The trimerization of ethylene by homogeneous catalysts has been demonstrated.

There are a wide range of applications for linear alpha olefins. The lower carbon numbers, 1-butene, 1-hexene and 1-octene can be used as comonomers in the production of polyethylene. High density polyethylene (HDPE) and linear low density polyethylene (LLDPE) can use approximately 2-4% and 8-10% of comonomers, respectively.

Another use of C₄-C₈ linear alpha olefins can be for production of linear aldehyde via oxo synthesis (hydroformylation) for later production of short-chain fatty acid, a carboxylic acid, by oxidation of an intermediate aldehyde, or linear alcohols for plasticizer application by hydrogenation of the aldehyde.

An application of 1-decene is in making polyalphaolefin synthetic lubricant base stock (PAO) and to make surfactants in a blend with higher linear alpha olefins.

C₁₀-C₁₄ linear alpha olefins can be used in making surfactants for aqueous detergent formulations. These carbon numbers can be reacted with benzene to make linear alkyl benzene (LAB), which can be further sulfonated to linear alkyl benzene sulfonate (LABS), a popular relatively low cost surfactant for household and industrial detergent applications.

Although some C₁₄ alpha olefin can be sold into aqueous detergent applications, C₁₄ has other applications such as being converted into chloroparaffins. A recent application of C₁₄ is as on-land drilling fluid base stock, replacing diesel or kerosene in that application. Although C₁₄ is more expensive than middle distillates, it has a significant advantage environmentally, being much more biodegradable and in handling the material, being much less irritating to skin and less toxic.

C₁₆-C₁₈ linear olefins find their primary application as the hydrophobes in oil-soluble surfactants and as lubricating fluids themselves. C₁₆-C₁₈ alpha or internal olefins are used as synthetic drilling fluid base for high value, primarily off-shore synthetic drilling fluids. The preferred materials for the synthetic drilling fluid application are linear internal olefins, which are primarily made by isomerizing linear alpha-olefins to an internal position. The higher internal olefins appear to form a more lubricious layer at the metal surface and are recognized as a better lubricant. Another application for C₁₆-C₁₈ olefins is in paper sizing. Linear alpha olefins are, once again, isomerized into linear internal olefins are then reacted with maleic anhydride to make an alkyl succinic anhydride (ASA), a popular paper sizing chemical.

C₂₀-C₃₀ linear alpha olefins production capacity can be 5-10% of the total production of a linear alpha olefin plant. These are used in a number of reactive and non-reactive applications, including as feedstocks to make heavy linear alkyl benzene (LAB) and low molecular weight polymers used to enhance properties of waxes.

The use of 1-hexene can be as a comonomer in production of polyethylene. High-density polyethylene (HDPE) and linear low-density polyethylene (LLDPE) use approximately 2-4% and 8-10% of comonomers, respectively.

Another use of 1-hexene is the production of the linear aldehyde heptanal via hydroformylation (oxo synthesis). Heptanal can be converted to the short-chain fatty acid heptanoic acid or the alcohol heptanol.

The effluent stream can then be contacted with a coolant stream. For example, the coolant stream can be directly mixed with the effluent stream. The coolant stream can be gaseous and/or liquid. The source of the coolant stream can be a product of the present oligomerization process itself. In other words, the coolant stream can be a recycled stream that is produced by the present process and then redirected for cooling purposes. For example, the coolant stream can comprise a mixture of hydrocarbons. For example, the coolant stream can comprise methane, ethylene, ethane, butane, hexane, or a combination comprising at least one of the foregoing. The coolant stream can also be imported from outside of the present process. For example, the coolant stream can be imported from a battery limit. A battery limit is generally described as a defined boundary between two areas of responsibility, for example, a flange on a pipe. Accordingly, a battery limit as described herein can refer to the coolant stream being imported from a flange on downstream equipment in the reactor.

Contact of the effluent stream with the coolant stream results in the cooling of the effluent stream. For example, the temperature of the effluent stream can be reduced by greater than or equal to 20%. This reduction in temperature results in the formation of condensate droplets within the gaseous effluent stream, for example, a two-phase flow can develop wherein liquid condensate droplets are formed in suspension within the gaseous effluent stream. The condensate droplets can then collide with, and dislodge, any polymer deposits present in the piping and downstream equipment. This is achieved, at least in part, by appropriate hydraulic design of the piping system. For example, the pipe diameter can be designed to accommodate a stream velocity that can maintain liquid droplets in suspension within the effluent stream and avoid any settling of the droplets.

The reduction in temperature of the effluent stream, when contacted with the coolant stream, can also serve to deactivate unreacted catalyst particles within the effluent stream. This can reduce the number of subsequently formed by-products and impurities within the effluent stream and any downstream processes.

The effluent stream can then be passed through additional downstream equipment, for example, a heat exchanger. The heat exchanger can utilize any desired cooling and/or heating means. For example, a heat exchanger fluid stream can be passed through the heat exchanger. Downstream heat exchangers, employed to cool the effluent stream, can remain substantially free of polymer deposits and experience a reduction in both duty and maintenance requirements. For example, a heat exchanger can experience a reduction of greater than or equal to 20% in duty requirements. A heat exchanger can also remain substantially free of polymer deposits, for example, less than or equal to 1 ppm polymer deposits can be present in the heat exchanger.

The present method of treating a hydrocarbon stream can also produce a product stream. For example, the product stream can comprise a mixture of hydrocarbons, for example, linear alpha olefins, for example, 1-hexene.

A more complete understanding of the components, processes, and apparatuses disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

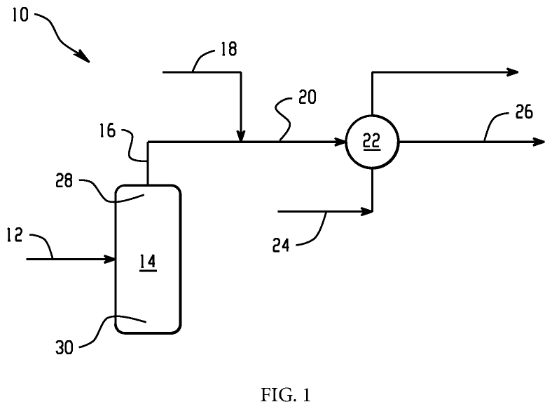

Referring now to FIG. 1, this schematic diagram represents a reactor scheme 10 in a method for treating a hydrocarbon stream. The reactor scheme 10 can include passing a feed stream 12 through a reactor 14. For example, the feed stream 12 can comprise a mixture of hydrocarbons, for example, linear alpha olefins, for example, ethylene. The feed stream 12 can also comprise a solvent, for example, toluene, as well as a catalyst, for example, a heterogeneous catalyst, for example, an oligomerization catalyst. The reactor 14 can be a multi-phase reactor, for example, an oligomerization reactor. Accordingly, an ethylene oligomerization reaction can occur within the reactor 14.

The reactor 14 can include a top portion 28 and a bottom portion 30. The reactor 14 can produce an effluent stream 16 which can be withdrawn from the top portion 28 of the reactor 14. For example, the effluent stream 16 can be gaseous and can comprise linear alpha olefins, solvent, catalyst, and dissolved polymer. For example, the dissolved polymer can be a by-product of the oligomerization reaction that occurs within the reactor 14. The effluent stream 16 can have a temperature of greater than or equal to 45° C. when withdrawn from the reactor. The effluent stream can be transferred from the reactor 14 to subsequent downstream equipment via piping.

The effluent stream 16 can then be contacted with a coolant stream 18. For example, the coolant stream 18 can be directly mixed with the effluent stream 16. The source of the coolant stream 18 can be a product of the present oligomerization process itself. In other words, the coolant stream 18 can be a recycled stream that is produced by the present process and then redirected for cooling purposes. For example, the coolant stream 18 can comprise a mixture of hydrocarbons.

Contact of the effluent stream 16 with the coolant stream 18 results in the cooling of the effluent stream 16. This in turn results in the formation of condensate droplets within the gaseous effluent stream 16. The condensate droplets can collide with any polymer by-product that is present within the effluent stream 16 as well as with any polymer present in the piping or equipment that the effluent stream 16 travels through. (Element 20 of FIG. 1 represents the effluent stream 16 after contact with coolant stream 18 and prior to passage through downstream equipment.)

The effluent stream 16 can then be passed through additional downstream equipment, for example, heat exchanger 22. Heat exchanger fluid stream 24 can pass through heat exchanger 22. The present method 10 of treating a hydrocarbon stream can also produce a product stream 26. For example, the product stream 26 can comprise a mixture of hydrocarbons, for example, linear alpha olefins, for example, 1-hexene.

The methods disclosed herein include(s) at least the following aspects:

Aspect 1: A method of treating a hydrocarbon stream, comprising: withdrawing an effluent stream comprising hydrocarbons and polymer from a reactor; contacting the effluent stream with a coolant stream; passing the effluent stream through a heat exchanger; wherein after passing the effluent stream through the heat exchanger, the heat exchanger is substantially free of polymer deposits.

Aspect 2: The method of Aspect 1, wherein the source of the effluent stream is a product of an ethylene oligomerization process.

Aspect 3: The method of any of the preceding aspects, wherein a temperature of the effluent stream is greater than or equal to 45° C. prior to contacting the coolant stream.

Aspect 4: The method of any of the preceding aspects, wherein the effluent stream comprises ethane, methane, ethylene, butene, hexene, toluene, octene, decene, catalyst particles, or a combination comprising at least one of the foregoing.

Aspect 5: The method of any of the preceding aspects, wherein the effluent stream comprises greater than or equal to 1 parts per million polymer.

Aspect 6: The method of any of the preceding aspects, wherein the effluent stream comprises 0% to 5% hexene.

Aspect 7: The method of any of the preceding aspects, wherein the effluent stream comprises 0 to 50 parts per million active catalyst particles.

Aspect 8: The method of any of the proceeding aspects, wherein the source of the coolant stream is a product of an ethylene oligomerization process and/or a product of a battery limit process.

Aspect 9: The method of any of the preceding aspects, wherein the source of the effluent stream and the coolant stream is a product of the same process.

Aspect 10: The method of any of the preceding aspects, wherein the coolant stream comprises a liquid.

Aspect 11: The method of any of the preceding aspects, wherein the coolant stream comprises methane, ethylene, ethane, butane, hexane, or a combination comprising at least one of the foregoing.

Aspect 12: The method of any of the preceding aspects, wherein the effluent stream and the coolant stream are directly mixed.

Aspect 13: The method of any of the preceding aspects, wherein the effluent stream is reduced in temperature by greater than or equal to 20% after contacting the coolant stream.

Aspect 14: The method of any of the preceding aspects, wherein the effluent stream and the coolant stream are contacted prior to passing through the heat exchanger.

Aspect 15: The method of any of the preceding aspects, wherein a duty of the heat exchanger is reduced by 20% as compared with a heat exchanger from a different method.

Aspect 16: The method of any of the preceding aspects, further comprising passing a fluid stream through the heat exchanger.

Aspect 17: The method of any of the preceding aspects, further comprising passing the effluent stream through a pipe.

Aspect 18: The method of Aspect 17, wherein after passing the effluent stream through the pipe, the pipe is substantially free of polymer deposits.

Aspect 19: The method of any of the preceding aspects, further comprising withdrawing a product stream from the heat exchanger.

Aspect 20: A method of treating a hydrocarbon stream, comprising: withdrawing an effluent stream comprising hydrocarbons and polymer from a reactor, wherein the effluent stream comprises ethane, methane, ethylene, butane, hexene, toluene, octene, decene, catalyst particles, or a combination comprising at least one of the foregoing; directly mixing the effluent stream with a coolant stream, wherein the coolant stream comprises methane, ethylene, ethane, butane, hexene, or a combination comprising at least one of the foregoing and wherein the effluent stream is reduced in temperature by greater than or equal to 20% after contacting the coolant stream; passing the effluent stream through a pipe, wherein after passing the effluent stream through the pipe, the pipe is substantially free of polymer deposits; passing the effluent stream through a heat exchanger, wherein after passing the effluent stream through the heat exchanger, the heat exchanger is substantially free of polymer deposits; and withdrawing a product stream from the heat exchanger; wherein the source of the effluent stream and the coolant stream is a product of the same ethylene oligomerization process.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of treating a hydrocarbon stream, comprising:
withdrawing an effluent stream comprising hydrocarbons and polymer from a reactor;
contacting the effluent stream with a coolant stream;
passing the effluent stream through a heat exchanger;
wherein after passing the effluent stream through the heat exchanger, the heat exchanger is substantially free of polymer deposits.

2. The method of claim 1, wherein the source of the effluent stream is a product of an ethylene oligomerization process.

3. The method of claim 1, wherein a temperature of the effluent stream is greater than or equal to 45° C. prior to contacting the coolant stream.

4. The method claim 1, wherein the effluent stream comprises ethane, methane, ethylene, butene, hexene, toluene, octene, decene, catalyst particles, or a combination comprising at least one of the foregoing.

5. The method of claim 1, wherein the effluent stream comprises greater than or equal to 1 parts per million polymer.

6. The method of claim 1, wherein the effluent stream comprises 0% to 5% hexene.

7. The method of claim 1, wherein the effluent stream comprises 0 to 50 parts per million active catalyst particles.

8. The method of claim 1, wherein the source of the coolant stream is a product of an ethylene oligomerization process and/or a product of a battery limit process.

9. The method of claim 1, wherein the source of the effluent stream and the coolant stream is a product of the same process.

10. The method of claim 1, wherein the coolant stream comprises a liquid.

11. The method of claim 1, wherein the coolant stream comprises methane, ethylene, ethane, butane, hexane, or a combination comprising at least one of the foregoing.

12. The method of claim 1, wherein the effluent stream and the coolant stream are directly mixed.

13. The method of claim 1, wherein the effluent stream is reduced in temperature by greater than or equal to 20% after contacting the coolant stream.

14. The method of claim 1, wherein the effluent stream and the coolant stream are contacted prior to passing through the heat exchanger.

15. The method of claim 1, wherein a duty of the heat exchanger is reduced by 20% as compared with a heat exchanger from a different method.

16. The method of claim 1, further comprising passing a fluid stream through the heat exchanger.

17. The method of claim 1, further comprising passing the effluent stream through a pipe.

18. The method of claim 17, wherein after passing the effluent stream through the pipe, the pipe is substantially free of polymer deposits.

19. The method of claim 1, further comprising withdrawing a product stream from the heat exchanger.

20. A method of treating a hydrocarbon stream, comprising:
withdrawing an effluent stream comprising hydrocarbons and polymer from a reactor, wherein the effluent stream comprises ethane, methane, ethylene, butane, hexene, toluene, octene, decene, catalyst particles, or a combination comprising at least one of the foregoing;
directly mixing the effluent stream with a coolant stream, wherein the coolant stream comprises methane, ethylene, ethane, butane, hexene, or a combination comprising at least one of the foregoing and wherein the effluent stream is reduced in temperature by greater than or equal to 20% after contacting the coolant stream;
passing the effluent stream through a pipe, wherein after passing the effluent stream through the pipe, the pipe is substantially free of polymer deposits;
passing the effluent stream through a heat exchanger, wherein after passing the effluent stream through the heat exchanger, the heat exchanger is substantially free of polymer deposits; and
withdrawing a product stream from the heat exchanger;
wherein the source of the effluent stream and the coolant stream is a product of the same ethylene oligomerization process.
